SEQUENCE LISTING (10 sequences)

SEQ ID NO: 1 - protein, 371 aa, Homo sapiens
MGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGVAAGSSSSVKWTEGQSNHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLK

SEQ ID NO: 2 - DNA, 3500 bp, Artificial Sequence
  expression plasmid
tgggcttttgctggccttttgctcacatgttcttgactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagacccccatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatc

SEQ ID NO: 3 - DNA, 1116 bp, Homo sapiens
atggacttcctcttgctgcaggacccggcttccacgtgtgtcccggagccggcgtctcagcacacgctccgctccgggcctgggtgcctacagcagccagagcagcagggagtccgggacccgggcggcatctgggccaagttaggcgccgccgaggccagcgctgaacgtctccagggccggaggagccgcggggcgtccgggtctgagccgcagcaaatgggctccgacgtgcgggacctgaacgcgctgctgcccgccgtcccctccctgggtggcggcggcggctgtgccctgcctgtgagcggcgcggcgcagtgggcgccggtgctggactttgcgcccccgggcgcttcggcttacgggtcgttgggcggccccgcgccgccaccggctccgccgccacccccgccgccgccgcctcactccttcatcaaacaggagccgagctggggcggcgcggagccgcacgaggagcagtgcctgagcgccttcactgtccacttttccggccagttcactggcacagccggagcctgtcgctacgggcccttcggtcctcctccgcccagccaggcgtcatccggccaggccaggatgtttcctaacgcgccctacctgcccagctgcctggagagccagcccgctattcgcaatcagggttacagcacggtcaccttcgacgggacgcccagctacggtcacacgccctcgcaccatgcggcgcagttccccaaccactcattcaagcatgaggatcccatgggccagcagggctcgctgggtgagcagcagtactcggtgccgcccccggtctatggctgccacacccccaccgacagctgcaccggcagccaggctttgctgctgaggacgccctacagcagtgacaatttataccaaatgacatcccagcttgaatgcatgacctggaatcagatgaacttaggagccaccttaaagggagttgctgctgggagctccagctcagtgaaatggacagaagggcagagcaaccacagcacagggtacgagagcgataaccacacaacgcccatcctctgcggagcccaatacagaatacacacgcacggtgtcttcagaggcattcagtga

SEQ ID NO: 4 - protein, 522 aa, Homo sapiens
MDFLLLQDPASTCVPEPASQHTLRSGPGCLQQPEQQGVRDPGGIWAKLGAAEASAERLQGRRSRGASGSEPQQMGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGVAAGSSSSVKWTEGQSNHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGKTSEKPFSCRWPSCQKKFARSDELVRHHNMHQRNMTKLQLAL

SEQ ID NO: 5 - protein, 630 aa, Homo sapiens
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQAPRRPLPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 6 - protein, 702 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNKLSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 7 - protein, 561 aa, Cytomegalovirus
MESRGRRCPEMISVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTERKTPRVTGGGAMAGASTSAGRKRKSASSATACTAGVMTRGRLKAESTVAPEEDTDEDSDNEIHNPAVFTWPPWQAGILARNLVPMVATVQGQNLKYQEFFWDANDIYRIFAELEGVWQPAAQPKRRRHRQDALPGPCIASTPKKHRG

SEQ ID NO: 8 - protein, 561 aa, Cytomegalovirus
MESRGRRCPEMISVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTERKTPRVTGGGAMAGASTSAGRNRKSASSATACTAGVMTRGRLKAESTVAPEEDTDEDSDNEIHNPAVFTWPPWQAGILARNLVPMVATVQGQNLKYQEFFWDANDIYRIFAELEGVWQPAAQPKRRRHRQDALPGPCIASTPKKHRG

SEQ ID NO: 9 - protein, 536 aa, Cytomegalovirus
MESRGRRCPEMISVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTERKTPRVTGGGAMAGASTSAGRNRKSASSATACTAGVMTRGRLKAESTVAPEEDTDEDSDNEIHNPAVFTWPPWQAGILARNLVPMVATVQGQNLKYQEFFWDANDIYRIFAELEGVWQPAAQ

SEQ ID NO: 10 - protein, 1356 aa, Homo sapiens
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIILRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

(19) United States
(12) Patent Application Publication
(10) Pub. No.: US 2023/0158133 A1
(43) Pub. Date: May 25, 2023

(54) WT1 TARGETING DNA VACCINE FOR COMBINATION THERAPY

(71) Applicant: VAXIMM AG, Basel (CH)

(72) Inventor: Heinz Lubenau, Neustadt an der Weinstrasse (DE)

(21) Appl. No.: 16/347,551

(22) PCT Filed: Nov. 3, 2017
(86) PCT No.: PCT/EP2017/078124

§ 371 Date: May 3, 2019

(30) Foreign Application Priority Data
Nov. 4, 2016 (EP) 16197322.7

(51) Int. Cl. A61K 39/00; A61P 35/02; A61K 39/395

(52) U.S. Cl. CPC A61K 39/001153; A61P 35/02; A61K 39/3955; A61K 39/001168; A61K 39/001182; A61K 39/001109; A61K 2039/522; A61K 2039/523; A61K 2039/53; A61K 2039/542; A61K 2039/545

(57) ABSTRACT

The present invention relates to an attenuated strain of Salmonella comprising at least one copy of a DNA molecule comprising an expression cassette encoding Wilms' Tumor Protein (WT1), for use in the treatment of cancer, wherein the treatment further comprises the administration of at least one checkpoint inhibitor, particularly selected from at least one antibody against PD-1, PD-L1, CTLA-4, IDO, OX-40, GITR, TIM-3, and LAG-3. The present invention further relates to a pharmaceutical composition comprising an attenuated strain of Salmonella comprising at least one copy of a DNA molecule comprising an expression cassette encoding WT1 for use in the treatment of cancer, wherein the treatment further comprises the administration of at least one checkpoint inhibitor, particularly selected from at least one antibody against PD-1, PD-L1, CTLA-4, IDO, OX-40, GITR, TIM-3, and LAG-3.

### FIELD OF THE INVENTION

The present invention relates to an attenuated strain of Salmonella comprising at least one copy of a DNA molecule comprising an expression cassette encoding Wilms' Tumor Protein (WT1), for use in the treatment of cancer, wherein the treatment further comprises the administration of at least one checkpoint inhibitor, particularly selected from at least one antibody against PD-1, PD-L1, CTLA-4, IDO, OX-40, GITR, TIM-3, and LAG-3. The present invention further relates to a pharmaceutical composition comprising an attenuated strain of Salmonella comprising at least one copy of a DNA molecule comprising an expression cassette encoding WT1 for use in the treatment of cancer, wherein the treatment further comprises the administration of at least one checkpoint inhibitor, particularly selected from at least one antibody against PD-1, PD-L1, CTLA-4, IDO, OX-40, GITR, TIM-3, and LAG-3.

### BACKGROUND OF THE INVENTION

Wilms' tumor gene 1 (WT1) encodes a zinc finger transcription factor involved in cell proliferation and differentiation. It is highly expressed in a wide variety of malignancies including several types of hematological malignancies and various solid tumors. In contrast, normal tissue expression of WT1 in adults is restricted to gonads, uterus, kidney, mesothelium and CD34⁺ progenitor cells in various types of tissues. WT1 was originally proposed as a tumor suppressor gene. However, more recent evidence points to oncogenic functions of this transcription factor; Wt-1 negatively affects differentiation and promotes proliferation of progenitor cells. Furthermore, overexpressed WT1 is immunogenic; WT1 specific T cells as well as IgG anti-WT1 antibodies have been observed in cancer patients. Thus, WT-1 is a promising candidate for the development of cancer vaccines.

Human clinical trials with WT1 vaccines based on HLA (human leukocyte antigen)-restricted WT1 peptide fragments have been reported. Osada et al., Clin Cancer Res 2009; 15:2789-2796, discloses a WT1-encoding Adenovirus Vaccine.

WO 2014/173542 discloses an attenuated strain of Salmonella comprising a recombinant DNA molecule encoding WT1 use in cancer immunotherapy. The attenuated Salmonella strain encoding WT1 was shown to exhibit antitumor activity in a mouse model challenged with murine leukemia cells. Thus, the attenuated Salmonella strain encoding WT1 has great potential as cancer vaccine for the treatment of these indications.

WO 2013/09189 discloses a method for growing attenuated mutant Salmonella typhi strains lacking galactose epimerase activity and harboring a recombinant DNA molecule.

The finding that tumors can be immunogenic has led to the development of a number of cancer immunotherapies designed to employ the immune system to selectively eliminate malignant cells while sparing normal tissue. However, survival benefits from vaccination against tumor antigens alone remain modest. Anti-cancer vaccines face numerous challenges, one of them being the immunosuppressive microenvironment. The abnormal tumor vasculature creates a hypoxic microenvironment that polarizes inflammatory cells toward immune suppression. Moreover, tumors systemically alter immune cells' proliferation, differentiation, and function via secretion of growth factors and cytokines.

For cure of cancer, complete eradication of cancer stem cells is of crucial importance. The numerous immune escape mechanisms of human tumors remain a major challenge in cancer immunotherapy. Thus, there exists a great need for improved cancer therapy approaches, which has not been met so far.

### OBJECTS OF THE INVENTION

In view of the prior art, it is an object of the present invention to provide novel cancer therapies. Such novel therapies would offer major advantages for improving the treatment options for cancer patients.

### SUMMARY OF THE INVENTION

In one aspect, the present invention relates to an attenuated strain of Salmonella comprising at least one copy of a DNA molecule comprising an expression cassette encoding Wilms' Tumor Protein (WT1), for use in the treatment of cancer, wherein the treatment further comprises the administration of at least one checkpoint inhibitor.

The combined treatment with an attenuated strain of Salmonella encoding WT1 and at least one checkpoint inhibitor was surprisingly found to exhibit a strong, and sustained anti-tumor effect. Surprisingly, it was observed that the combined administration of an attenuated strain of Salmonella encoding WT1 with either of the checkpoint inhibitors anti-PD-L1 and anti-CTLA-4 has a synergistic effect on overall survival.

Without wishing to be bound by theory, it is believed that VXM06 is able to generate WT-1 specific effector T-cells which might become inactivated by the tumor microenvironment. Checkpoint inhibitor monoclonal antibodies target structures on immune cells or tumor cells, respectively, and are therefore reported to counterbalance or prevent T-cell inhibitory effects.

In particular embodiments, the at least one checkpoint inhibitor is selected from at least one antibody against PD-1, PD-L1, CTLA-4, IDO, OX-40, GITR, TIM-3, and LAG-3.

In particular embodiments the attenuated strain of Salmonella is of the species Salmonella enterica. Particularly, the attenuated strain of Salmonella is Salmonella typhi Ty21 a.

In particular embodiments, the expression cassette is a eukaryotic expression cassette. Particularly, the expression cassette comprises a CMV promoter.

In particular embodiments, WT1 is selected from the group consisting of human WT1 having the amino acid sequence as found in SEQ ID NO 4 and a protein that shares at least about 80% sequence identity therewith. Particularly, WT1 is truncated, more particularly the zinc finger domain of WT1 is deleted. In particular such embodiments, WT1 is selected from the group consisting of WT1 having the amino acid sequence as found in SEQ ID NO 1 and a protein that shares at least 80% sequence identity therewith.

In particular embodiments, the DNA molecule comprises the kanamycin antibiotic resistance gene, the pMB1 ori, and a CMV promoter. Particularly, the DNA molecule comprises the DNA sequence as found in SEQ ID NO 2.

In particular embodiments, the attenuated strain of Salmonella is administered simultaneously with, prior to or after said at least one checkpoint inhibitor.

In particular embodiments, the treatment is accompanied by chemotherapy, radiotherapy or biological cancer therapy, particularly wherein the attenuated strain of Salmonella is administered before, during or after the chemotherapy or the radiotherapy treatment cycle or the biological cancer therapy, or before and during the chemotherapy or the radiotherapy treatment cycle or the biological cancer therapy.

In particular embodiments, the biological cancer therapy comprises administration of one or more further attenuated strain(s) of Salmonella comprising at least one copy of a DNA molecule comprising an expression cassette encoding a tumor antigen and/or a tumor stroma antigen. In particular such embodiments, said one or more further attenuated strain(s) of Salmonella is/are Salmonella typhi Ty21a comprising a eukaryotic expression cassette. Particularly, said tumor antigen is selected from the group consisting of Mesothelin (MSLN), CEA, CMV pp65, preferably said tumor antigen is selected from the group (a) Mesothelin (MSLN), particularly MSLN having the amino acid sequence as found in SEQ ID NO 5 and a protein that shares at least about 80% sequence identity therewith, (b) CEA, particularly CEA having the amino acid sequence as found in SEQ ID NO 6 and a protein that shares at least about 80% sequence identity therewith, and (c) CMV pp65, particularly CMV pp65 having the amino acid sequence as found in SEQ ID NO 7 and a protein that shares at least about 80% sequence identity therewith, CMV pp65 having the amino acid sequence as found in SEQ ID NO 8 and a protein that shares at least about 80% sequence identity therewith, and CMV pp65 having the amino acid sequence as found in SEQ ID NO 9 and a protein that shares at least about 80% sequence identity therewith. Particularly, said tumor stroma antigen is selected from the group consisting of a VEGF receptor protein and human fibroblast activation protein (FAP), wherein preferably the VEGF receptor protein is VEGFR-2 more preferably human VEGFR-2 and even more preferably human VEGFR-2 having the amino acid sequence as found in SEQ ID NO 10 or a protein that shares at least about 80% sequence identity therewith.

In particular embodiments, the attenuated strain of Salmonella is administered orally.

In particular embodiments, the cancer is selected from leukemia, particularly from acute myeloid leukemia (AML) and acute lymphoid leukemia (ALL), from multiple myeloma, and from solid tumors, particularly from lung cancer, breast cancer, esophageal, colon, colorectal, gastric, cholangioductal, pancreatic cancer, glioblastoma, head and neck cancer, synovial sarcoma, angiosarcoma, osteosarcoma, thyroid cancer, cervical, endometrial, ovarian cancer, neuroblastoma, rhabdomyosarcoma, and prostate cancer.

In particular embodiments, the single dose of the attenuated strain of Salmonella comprises from about 10⁵ to about 10¹¹, particularly from about 10⁶ to about 10¹⁰, more particularly from about 10⁶ to about 10⁹, more particularly from about 10⁶ to about 10⁸, most particularly from about 10⁶ to about 10⁷ colony forming units (CFU).

In particular embodiments, the treatment is individualized cancer immunotherapy comprising the step of assessing the WT1 expression and/or the pre-immune response against WT1 in a patient.

In a further aspect, the present invention relates to a pharmaceutical composition comprising an attenuated strain of Salmonella comprising at least one copy of a DNA molecule comprising an expression cassette encoding WT1 for use in the treatment of cancer, wherein the treatment further comprises the administration of at least one checkpoint inhibitor. Particularly, the at least one checkpoint inhibitor is selected from at least one antibody against PD-1, PD-L1, CTLA-4, IDO, OX-40, GITR, TIM-3, and LAG-3.

In particular embodiments, the attenuated strain of Salmonella is Salmonella typhi Ty21a, the expression cassette is a eukaryotic expression cassette, and WT1 is selected from the group consisting of human WT1 having the amino acid sequence as found in SEQ ID NO 1 and a protein that shares at least about 80% sequence identity therewith.

Particularly, the eukaryotic expression cassette comprises a CMV promoter. Particularly, human WT1 has the amino acid sequence as found in SEQ ID NO 1.

### DETAILED DESCRIPTION OF THE INVENTION

The present invention may be understood more readily by reference to the following detailed description of the invention and the examples included therein.

In one aspect, the present invention relates to an attenuated strain of Salmonella comprising at least one copy of a DNA molecule comprising an expression cassette encoding Wilms' Tumor Protein (WT1) for use in the treatment of cancer, wherein the treatment further comprises the administration of at least one checkpoint inhibitor.

The zinc finger transcription factor Wilms' tumor protein 1 is encoded by the WT1 gene. It contains four zinc finger motifs at the C-terminus and a proline/glutamine-rich DNA-binding domain at the N-terminus. Multiple transcript variants, resulting from alternative splicing at two coding exons, have been well characterized. WT1 plays an essential role in the development of the urogenital system and is involved in cell proliferation and differentiation. The WT1 gene was isolated as the gene responsible for a childhood renal neoplasm, Wilms' tumor. It is highly expressed in a wide variety of malignancies including several types of hematological malignancies and various solid tumors. In contrast, normal tissue expression of WT1 in adults is restricted to gonads, uterus, kidney, mesothelium and progenitor cells in various types of tissues. Due to its expression profile, its oncogenic functions and its immunogenic potential, the tumor antigen WT1 is a promising candidate for the development of cancer vaccines.

According to the invention, the attenuated Salmonella strain functions as the bacterial carrier of the recombinant DNA molecule comprising an expression cassette encoding WT1 for the delivery of said recombinant DNA molecule into a target cell. Such a delivery vector comprising a DNA molecule encoding a heterologous antigen, such as WT1 — a tumor antigen, is termed DNA vaccine.

In the context of the present invention, the term “vaccine” refers to an agent which is able to induce an immune response in a subject upon administration. A vaccine can preferably prevent, ameliorate or treat a disease.

The live attenuated Salmonella strain according to the present invention stably carries a recombinant DNA molecule encoding WT1. It can be used as a vehicle for the oral delivery of this recombinant DNA molecule.

Genetic immunization might be advantageous over conventional vaccination. The target DNA can be detected for a considerable period of time thus acting as a depot of the antigen. Sequence motifs in some plasmids, like GpC islands, are immunostimulatory and can function as adjuvants furthered by the immunostimulation due to LPS and other bacterial components.

In contrast to peptide vaccines that can only mediate immunity against a small fragment of the WT1 protein, genetic vaccination may result in immunity against a wide variety of epitopes present over the whole length of the encoded WT1 protein.

Apart from that, WT1 peptide vaccines, which have been used in clinical trials for the most part, have limited application due to HLA restriction of the peptides, i.e. their binding capacity to HLA molecules of antigen presenting cells (APCs). In contrast, the DNA vaccine of the present invention is not HLA restricted. Furthermore, the peptide fragment encoded might not be present in the patient's tumor in spite of positivity for WT-1. As VXM06 encodes the full-length protein WT-1 except for the zinc finger domain of WT1, the peptide fragments presented to the immune system are produced by the patient.

Live attenuated Salmonella vectors produce their own immunomodulatory factors such as lipopolysaccharides (LPS) in situ which may constitute an advantage over other forms of administration such as microencapsulation. Moreover, the mucosal vaccine according to the present invention has an intra-lymphatic mode of action, which proves to be of benefit. After ingestion of the attenuated vaccine according to the present invention, macrophages and other cells in Peyer's patches of the gut are invaded by the modified bacteria. The bacteria are taken up by these phagocytic cells. Due to their attenuating mutations, bacteria of the S. typhi Ty21 strain are not able to persist in these phagocytic cells but die at this time point. The recombinant DNA molecules are released and subsequently transferred into the cytosol of the phagocytic immune cells, either via a specific transport system or by endosomal leakage. Finally, the recombinant DNA molecules enter the nucleus, where they are transcribed, leading to massive WT1 expression in the cytosol of the phagocytic cells. The infected cells undergo apoptosis, loaded with the WT1 antigen, and are taken up and processed by the gut's immune system. The danger signals of the bacterial infection serve as a strong adjuvant in this process, leading to a strong target antigen specific CD8+T-cell and antibody response at the level of both systemic and mucosal compartments. The immune response peaks around ten days after vaccination. The lack of anti-carrier response allows boosting with the same vaccine over many times.

In the context of the present invention, the term “attenuated” refers to a bacterial strain of reduced virulence compared to the parental bacterial strain, not harboring the attenuating mutation. Attenuated bacterial strains have preferably lost their virulence but retained their ability to induce protective immunity. Attenuation can be accomplished by deletion of various genes, including virulence, regulatory, and metabolic genes. Attenuated bacteria may be found naturally or they may be produced artificially in the laboratory, for example by adaptation to a new medium or cell culture or they may be produced by recombinant DNA technology. Administration of about 10¹¹ CFU of the attenuated strain of Salmonella according to the present invention preferably causes Salmonellosis in less than 5%, more preferably less than 1%, most preferably less than 1% of subjects.

In the context of the present invention, the term “comprises” or “comprising” means “including, but not limited to”. The term is intended to be open-ended, to specify the presence of any stated features, elements, integers, steps or components, but not to preclude the presence or addition of one or more other features, elements, integers, steps, components or groups thereof. The term “comprising” thus includes the more restrictive terms “consisting of” and “essentially consisting of”. In one embodiment, the term “comprising” as used throughout the application and in particular within the claims may be replaced by the term “consisting of”.

The DNA molecule comprising an expression cassette encoding WT1 is suitably a recombinant DNA molecule, i.e. an engineered DNA construct, preferably composed of DNA pieces of different origin. The DNA molecule can be a linear nucleic acid, or preferably, a circular DNA plasmid generated by introducing an open reading frame encoding WT1 into an expression vector plasmid.

In the context of the present invention, the term “expression cassette” refers to a nucleic acid unit comprising at least one open reading frame (ORF) under the control of regulatory sequences controlling its expression. Expression cassettes can preferably mediate transcription of the included open reading frame encoding a tumor antigen, such as WT1, in a target cell. Expression cassettes typically comprise a promoter, at least one open reading frame and a transcription termination signal.

In particular embodiments, the at least one checkpoint inhibitor is selected from at least one antibody against PD-1, PD-L1, CTLA-4, IDO, OX-40, GITR, TIM-3, and LAG-3. Preferably the at least one checkpoint inhibitor is at least one antibody against PD-1, PD-L1 or CTLA-4, or a combination thereof, more preferably the at least one checkpoint inhibitor is at least one antibody against PD-L1 or CTLA-4, or a combination thereof.

An important part of the immune system is its ability to tell between normal cells in the body and those it sees as “foreign.” This lets the immune system attack the foreign cells while leaving the normal cells alone. To do this, it uses “checkpoints”—molecules on certain immune cells that need to be activated (or inactivated) to start an immune response.

Cancer cells sometimes find ways to use these checkpoints to avoid being attacked by the immune system. But drugs that target these checkpoints hold a lot of promise as cancer treatments. E.g. PD-1 is a checkpoint protein on immune cells called T cells. It normally acts as a type of “off switch” that helps keep the T cells from attacking other cells in the body. It does this when it attaches to PD-L1, a protein on some normal (and cancer) cells. When PD-1 binds to PD-L1, it basically tells the T cell to leave the other cell alone. Some cancer cells have large amounts of PD-L1, which helps them evade immune attack.

Monoclonal antibodies that target either PD-1 or PD-L1 have shown to be promising candidates for treating certain cancers. Surprisingly, it was found that these checkpoint inhibitors can boost the immune response against WT-1 expressing cancer cells that is mediated by the attenuated Salmonella strain encoding WT1.

In particular embodiments the attenuated strain of Salmonella is of the species Salmonella enterica. Attenuated derivatives of Salmonella enterica are attractive vehicles for the delivery of heterologous antigens to the mammalian immune system, since S. enterica strains can potentially be delivered via mucosal routes of immunization, i.e. orally or nasally, which offers advantages of simplicity and safety compared to parenteral administration. Furthermore, Salmonella strains elicit strong humoral and cellular immune responses at the level of both systemic and mucosal compartments. Batch preparation costs are low and formulations of live bacterial vaccines are highly stable. Attenuation can be accomplished by deletion of various genes, including virulence, regulatory, and metabolic genes.

Several Salmonella typhimurium strains attenuated by aro mutations have been shown to be safe and effective delivery vehicles for heterologous antigens in animal models.

In particular embodiments, the attenuated strain of Salmonella and the at least one further attenuated strain of Salmonella are Salmonella typhi Ty21a. The live, attenuated S. typhi Ty21 a strain is the active component of Typhoral Le, also known as Vivotif® (manufactured by Berna Biotech Ltd., a Crucell Company, Switzerland). It is currently the only licensed live oral vaccine against typhoid fever. This vaccine has been extensively tested and has proved to be safe regarding patient toxicity as well as transmission to third parties (Wandan et al., J. Infectious Diseases 1982, 145:292-295). The vaccine is licensed in more than 40 countries and has been used in millions of individuals including thousands of children for prophylactic vaccination against typhoid fever. It has an unparalleled safety track record. There is no data available indicating that S. typhi Ty21a is able to enter the bloodstream systemically. The live attenuated Salmonella typhi Ty21a vaccine strain thus allows specific targeting of the immune system in the gut, while being safe and well-tolerated. The Marketing Authorization number of Typhoral L® is PL 15747/0001 dated 16 December 1996. One dose of vaccine contains at least 2×10⁹ viable S. typhi Ty21a colony forming units and at least 5×10⁹ non-viable S. typhi Ty21a cells.

This well-tolerated, live oral vaccine against typhoid fever was derived by chemical mutagenesis of the wild-type virulent bacterial isolate S. typhi Ty2 and harbors a loss-of-function mutation in the galE gene resulting in its inability to metabolize galactose. The attenuated bacterial strain is also not able to reduce sulfate to sulfide which differentiates it from the wild-type Salmonella typhi Ty2 strain. With regard to its serological characteristics, the Salmonella typhi Ty21 a strain contains the O9-antigen which is a polysaccharide of the outer membrane of the bacteria and lacks the O5-antigen which is in turn a characteristic component of Salmonella typhimurium. This serological characteristic supports the rationale for including the respective test in a panel of identity tests for batch release.

In particular embodiments, the expression cassette is a eukaryotic expression cassette. Particularly, the expression cassette comprises a CMV promoter. In the context of the present invention, the term “eukaryotic expression cassette” refers to an expression cassette which allows for expression of the open reading frame in a eukaryotic cell. It has been shown that the amount of heterologous antigen required to induce an adequate immune response may be toxic for the bacterium and may result in cell death, over-attenuation or loss of expression of the heterologous antigen. Using a eukaryotic expression cassette that is not expressed in the bacterial vector but only in the target cell may overcome this toxicity problem and the protein expressed typically exhibits a eukaryotic glycosylation pattern.

A eukaryotic expression cassette comprises regulatory sequences that are able to control the expression of an open reading frame in a eukaryotic cell, preferably a promoter and a polyadenylation signal. Promoters and polyadenylation signals included in the recombinant DNA molecules comprised by the attenuated strain of Salmonella of the present invention are preferably selected to be functional within the cells of the subject to be immunized. Examples of suitable promoters, especially for the production of a DNA vaccine for humans, include but are not limited to promoters from Cytomegalovirus (CMV), such as the strong CMV immediate early promoter, Simian Virus 40 (SV40), Mouse Mammary Tumor Virus (MMTV), Human Immunodeficiency Virus (HIV), such as the HIV Long Terminal Repeat (LTR) promoter, Moloney virus, Epstein Barr Virus (EBV), and from Rous Sarcoma Virus (RSV), the synthetic CAG promoter composed of the CMV early enhancer element, the promoter, the first exon and the first intron of chicken beta-actin gene and the splice acceptor of the rabbit beta globin gene, as well as promoters from human genes such as human actin, human myosin, human hemoglobin, human muscle creatine, and human metallothionein. In a particular embodiment, the eukaryotic expression cassette contains the CMV promoter. In the context of the present invention, the term “CMV promoter” refers to the strong immediate-early cytomegalovirus promoter.

Examples of suitable polyadenylation signals, especially for the production of a DNA vaccine for humans, include but are not limited to the bovine growth hormone (BGH) polyadenylation site, SV40 polyadenylation signals and LTR polyadenylation signals. In a particular embodiment, the eukaryotic expression cassette included in the recombinant DNA molecule comprised by the attenuated strain of Salmonella of the present invention comprises the BGH polyadenylation site.

In addition to the regulatory elements required for expression of WT1, like a promoter and a polyadenylation signal, other elements can also be included in the recombinant DNA molecule. Such additional elements include enhancers. The enhancer can be, for example, the enhancer of human actin, human myosin, human hemoglobin, human muscle creatine and viral enhancers such as those from CMV, RSV and EBV.

Regulatory sequences and codons are generally species dependent, so in order to maximize protein production, the regulatory sequences and codons are preferably selected to be effective in the species to be immunized. The person skilled in the art can produce recombinant DNA molecules that are functional in a given subject species.

In particular embodiments, WT1 is selected from the group consisting of human WT1 having the amino acid sequence as found in SEQ ID NO 4 and a protein that shares at least about 80% sequence identity therewith. Particularly, WT1 is truncated, more particularly the zinc finger domain of WT1 is deleted. In particular such embodiments, WT1 is selected from the group consisting of WT1 having the amino acid sequence as found in SEQ ID NO 1 and a protein that shares at least about 80% sequence identity therewith. Particularly, WT1 has the amino acid sequence as found in SEQ ID NO 1.

The zinc finger domain at the C-terminus of WT1 comprises four zinc finger motifs. Truncated WT1 of the amino acid sequence as found in SEQ ID NO 1 represents amino acids 74 to 444 of UniProt ref P19544-7. Deletion of the zinc finger domain minimizes the risk of immunological cross reactivity with other zinc finger containing transcription factors. Furthermore, truncated WT1 lacking the zinc finger domain has greater immunogenic potential than full-length WT1. In addition, deletion of the zinc finger motifs, which are essential for DNA binding, abrogates the oncogenic potential of WT1, thus minimizing the risk of oncogenesis.

In this context, the term “about” or “approximately” means within 80% to 120%, alternatively within 90% to 110%, including within 95% to 105% of a given value or range.

In the context of the present invention, the term “protein that shares at least about 80% sequence identity with a given protein”, e.g., human WT1 having the amino acid sequence as found in SEQ ID NO 1 or SEQ ID NO 4 refers to a protein that may differ in the amino acid sequence and/or the nucleic acid sequence encoding the amino acid sequence of said reference protein, e.g., human WT1 having the amino acid sequence of SEQ ID NO 1 or SEQ ID NO 4, respectively. The protein may be of natural origin, e.g. a mutant version of a wild-type protein, e.g. a mutant version of a wild type WT1, or a homolog of a different species, or an engineered protein, e.g., engineered WT1. It is known that the usage of codons is different between species. Thus, when expressing a heterologous protein in a target cell, it may be necessary, or at least helpful, to adapt the nucleic acid sequence to the codon usage of the target cell. Methods for designing and constructing derivatives of a given protein are well known to anyone of ordinary skill in the art.

The protein that shares at least about 80% sequence identity with a given protein, e.g., human WT1 having the amino acid sequence as found in SEQ ID NO 1 or SEQ ID NO 4, may contain one or more mutations comprising an addition, a deletion and/or a substitution of one or more amino acids in comparison to the reference protein, e.g., WT1 having the amino acid sequence of SEQ ID NO 1 or SEQ ID NO 4, respectively. According to the teaching of the present invention, said deleted, added and/or substituted amino acids may be consecutive amino acids or may be interspersed over the length of the amino acid sequence of the protein that shares at least about 80% sequence identity with a reference protein, e.g., human WT1 having the amino acid sequence as found in SEQ ID NO 1 or SEQ ID NO 4, respectively. According to the teaching of the present invention, any number of amino acids may be added, deleted, and/or substitutes, as long as the amino acid sequence identity with the reference protein is at least about 80% and the mutated protein is immunogenic. Preferably, the immunogenicity of the protein which shares at least about 80% sequence identity with a given reference protein, e.g., human WT1 having the amino acid sequence as found in SEQ ID NO 1 or SEQ ID NO 4, is reduced by less than 50%, less than 40%, less than 30%, less than 20%, less than 10%, less than 5% or less than 1% compared to said reference protein, e.g., WT1 having the amino acid sequence as found in SEQ ID NO 1 or SEQ ID NO 4, respectively, as measured by ELISA. Methods for designing and constructing protein homologues and for testing such homologues for their immunogenic potential are well known to anyone of ordinary skill in the art. In particular embodiments, the sequence identity with the reference protein, e.g., WT1 having the amino acid sequence of SEQ ID NO 1 or SEQ ID NO 4, is at least about 80%, at least about 85%, at least about 90%, or most particularly at least about 95%. Methods and algorithms for determining sequence identity including the comparison of a parental protein and its derivative having deletions, additions and/or substitutions relative to a parental sequence, are well known to the practitioner of ordinary skill in the art. On the DNA level, the nucleic acid sequences encoding the protein that shares at least about 80% sequence identity with a given reference protein, e.g., human WT1 having the amino acid sequence as found in SEQ ID NO 1 or SEQ ID NO 4, may differ to a larger extent due to the degeneracy of the genetic code.

In the context of the present invention, the term “truncated WT1” refers to WT1 harboring one or more deletions of one amino acid or more than one consecutive amino acids each. According to the teaching of the present invention, any number of amino acids may be deleted, as long as the mutated protein is immunogenic. Preferably, the immunogenicity of the truncated WT1 protein is reduced by less than 50%, less than 40%, less than 30%, less than 20%, less than 10%, less than 5% or less than 1% compared to full length WT1 having the amino acid sequence as found in SEQ ID NO 4 (UniProt ref P19544-7), as measured by ELISA. Methods for designing and constructing protein homologues and for testing such homologues for their immunogenic potential are well known to anyone of ordinary skill in the art. In particular embodiments, less than 500, less than 400, less than 350, less than 300, less than 250, less than 200, less than 175, less than 150, less than 125, less than 100, less than 75, less than 50 or less than 25 amino acids are deleted from human full length WT1 having the amino acid sequence as found in SEQ ID NO 4 (UniProt ref P19544-7) or a protein that shares at least about 80% sequence identity therewith. Particularly, truncated WT1 has the zinc finger domain of WT1 deleted and is preferably selected from the group consisting of WT1 having the amino acid sequence as found in SEQ ID NO 1 and a protein that shares at least about 80% sequence identity therewith.

In particular embodiments, the DNA molecule comprises the kanamycin antibiotic resistance gene, the pMB1 ori, and a CMV promoter. In particular embodiments, the recombinant DNA molecule is derived from commercially available pVAX1TM expression plasmid (Invitrogen, San Diego, Calif.). This expression vector was modified by replacing the high copy pUC origin of replication by the low copy pMB1 origin of replication of pBR322. The low copy modification was made in order to reduce the metabolic burden and to render the construct more stable. The generated expression vector backbone was designated pVAX10.

In particular embodiments, the DNA molecule comprises the DNA sequence as found in SEQ ID NO 2 (vector backbone pVAX10).

The ORF having the nucleic acid sequence as found in SEQ ID NO 3 encodes human WT1, wherein the zinc finger domain is deleted. Inserting this ORF into the expression vector backbone (pVAX10) via NheI/XhoI yielded the expression plasmid pVAX10.hWT1. The expression plasmid pVAX10.hWT1 is schematically depicted in FIG. 11. The DNA vaccine comprising the attenuated Salmonella strain Ty21a harboring the expression plasmid pVAX10.hWT1 is designated VXM06.

In particular embodiments, the attenuated strain of Salmonella is administered simultaneously with, prior to or after said at least one checkpoint inhibitor.

In the context of the present invention, the term “simultaneously with” means administration of the attenuated strain of Salmonella encoding WT1 and the at least one checkpoint inhibitor on the same day, more particularly within 12 hours, more particularly within 2 hours.

In particular embodiments, administration of the attenuated Salmonella strain encoding WT1 and the at least one checkpoint inhibitor occurs within eight consecutive weeks, more particularly within three to six consecutive weeks. The attenuated Salmonella strain encoding WT1 and the at least one checkpoint inhibitor may be administered via the same route or via different routes.

In particular embodiments, the treatment is accompanied by chemotherapy, radiotherapy or biological cancer therapy. For cure of cancer, complete eradication of cancer stem cells may be essential. For maximal efficacy, a combination of different therapy approaches may be beneficial.

In the context of the present invention, the term “biological cancer therapy” refers to cancer therapy involving the use of living organisms including viruses, substances derived from living organisms or laboratory-produced versions of such substances. Some biological therapies for cancer aim at stimulating the body's immune system to act against cancer cells (so called biological cancer immunotherapy). Biological cancer therapy approaches include the delivery of tumor antigens and tumor stroma antigens, e.g. by Salmonella based DNA vaccines, particularly S. typhi Ty21a based DNA vaccines, delivery of therapeutic antibodies as drugs, administration of immunostimulatory cytokines and administration of immune cells, including engineered T cells. Therapeutic antibodies include antibodies targeting tumor antigens or tumor stroma antigens.

In particular embodiments, the biological cancer therapy comprises administration of one or more further attenuated strain(s) of Salmonella comprising at least one copy of a DNA molecule comprising an expression cassette encoding a tumor antigen and/or a tumor stroma antigen. In particular such embodiments, said one or more further attenuated strain(s) of Salmonella is/are Salmonella typhi Ty21a comprising a eukaryotic expression cassette. Particularly, said tumor antigen is selected from the group consisting of Mesothelin (MSLN), CEA, and CMV pp65, preferably said tumor antigen is selected from the group consisting of (a) Mesothelin (MSLN), particularly MSLN having the amino acid sequence as found in SEQ ID NO 5 and a protein that shares at least about 80% sequence identity therewith, (b) CEA, particularly CEA having the amino acid sequence as found in SEQ ID NO 6 and a protein that shares at least about 80% sequence identity therewith, and (c) CMV pp65, particularly CMV pp65 having the amino acid sequence as found in SEQ ID NO 7 and a protein that shares at least about 80% sequence identity therewith, CMV pp65 having the amino acid sequence as found in SEQ ID NO 8 and a protein that shares at least about 80% sequence identity therewith, and CMV pp65 having the amino acid sequence as found in SEQ ID NO 9 and a protein that shares at least about 80% sequence identity therewith. Particularly, said tumor stroma antigen is selected from the group consisting of a VEGF receptor protein and human fibroblast activation protein (FAP), wherein preferably the VEGF receptor protein is VEGFR-2 more preferably human VEGFR-2 and even more preferably human VEGFR-2 having the amino acid sequence as found in SEQ ID NO 10 or a protein that shares at least about 80% sequence identity therewith.

Chemotherapeutic agents that may be used in combination with the attenuated mutant strain of Salmonella of the present invention may be, for example: gemcitabine, amifostine (ethyol), cabazitaxel, cisplatin, dacarbazine (DTIC), dactinomycin, docetaxel, mechlorethamine, streptozocin, cyclophosphamide, carrnustine (BCNU), lomustine (CCNU), doxorubicin (adriamycin), doxorubicin lipo (doxil), folinic acid, gemcitabine (gemzar), daunorubicin, daunorubicin lipo (daunoxome), procarbazine, ketokonazole, mitomycin, cytarabine, etoposide, methotrexate, 5-fluorouracil (5-FU), vinblastine, vincristine, bleomycin, paclitaxel (taxol), docetaxel (taxotere), aldesleukin, asparaginase, busulfan, carboplatin, cladribine, camptothecin, CPT-11, 10-hydroxy-7-ethyl-camptothecin (SN38), dacarbazine, floxuridine, fludarabine, hydroxyurea, ifosfamide, idarubicin, mesna, interferon alpha, interferon beta, irinotecan, mitoxantrone, topotecan, leuprolide, megestrol, melphalan, mercaptopurine, oxaliplatin, plicamycin, mitotane, pegaspargase, pentostatin, pipobroman, plicamycin, streptozocin, tamoxifen, teniposide, testolactone, thioguanine, thiotepa, uracil mustard, vinorelbine, chlorambucil, bortezomibe, thalidomide, lenalidomide and combinations thereof.

Most preferred chemotherapeutic agents according to the invention in combination with VXM06 and the at least one checkpoint inhibitor are cabazitaxel, carboplatin, oxaliplatin, cisplatin, cyclophosphamide, daunorubicine, idarubicine, epirubicine, etoposide, docetaxel, gemcitabine, doxorubicin, paclitaxel (taxol), irinotecan, vincristine, vinblastine, vinorelbin, folinic acid, 5-fluorouracil, ifosfamide and bleomycin, especially gemcitabine.

Particularly, the attenuated strain of Salmonella is administered before, during or after the chemotherapy or the radiotherapy treatment cycle or the biological cancer therapy. In other particular embodiments, the attenuated strain of Salmonella is administered before and during the chemotherapy or the radiotherapy treatment cycle or the biological cancer therapy.

In particular embodiments, the attenuated strain of Salmonella is administered orally. Oral administration is simpler, safer and more comfortable than parenteral administration. However, it has to be noted that the attenuated strain of Salmonella encoding WT1 may also be administered by any other suitable route. Preferably, a therapeutically effective dose is administered to the subject, and this dose depends on the particular application, the type of malignancy, the subject's weight, age, sex and state of health, the manner of administration and the formulation, etc. Administration may be single or multiple, as required.

The attenuated strain of Salmonella encoding WT1 may be provided in the form of a solution, a suspension, a lyophilisate, an enteric coated capsule, or any other suitable form. Typically, the attenuated strain of Salmonella is formulated as drinking solution. This embodiment offers the advantage of improved patient compliance. Preferably, the drinking solution comprises means to neutralize gastric acids at least to a certain degree, i.e. to bring the pH of the gastric juice closer to a pH of 7. Preferably, the drinking solution is a buffered suspension comprising the attenuated strain of Salmonella encoding WT1. In a particular embodiment, the buffered suspension is obtained by suspending the attenuated strain of Salmonella in a suitable buffer, preferably containing 2.6 g sodium hydrogen carbonate, 1.7 g L-ascorbic acid, 0.2 g lactose monohydrate and 100 ml of drinking water.

The at least one checkpoint inhibitor is preferably administered in the approved galenic formulation of the commercial product.

In particular embodiments, the cancer is selected from leukemia, particularly from acute myeloid leukemia (AML) and acute lymphoid leukemia (ALL), from multiple myeloma, and from solid tumors, particularly from lung cancer, breast cancer, esophageal, colon, colorectal, gastric, cholangioductal, pancreatic cancer, glioblastoma, head and neck cancer, synovial sarcoma, angiosarcoma, osteosarcoma, thyroid cancer, cervical, endometrial, ovarian cancer, neuroblastoma, rhabdomyosarcoma, and prostate cancer.

The attenuated strain of Salmonella encoding WT1 is surprisingly effective at relatively low doses. Furthermore, combined administration of the attenuated strain of Salmonella encoding WT1 together with at least one checkpoint inhibitor surprisingly shows synergistic effects on WT1-specific T-cell responses, tumor growth and/or overall survival at relatively low doses of the attenuated strain of Salmonella encoding WT1. Administration of low doses of live bacterial vaccines minimizes the risk of excretion and thus of transmission to third parties.

In particular embodiments, the single dose of the attenuated strain of Salmonella comprises from about 10⁵ to about 10¹¹, particularly from about 10⁶ to about 10¹⁰, more particularly from about 10⁶ to about 10⁹, more particularly from about 10⁶ to about 10⁸, most particularly from about 10⁶ to about 10⁷ colony forming units (CFU).

In this context, the term “about” or “approximately” means within a factor of 3, alternatively within a factor of 2, including within a factor of 1.5 of a given value or range.

In particular embodiments, the treatment is individualized cancer immunotherapy comprising the step of assessing the expression of WT1 and/or the pre-immune response against WT1 in a patient. The patient's WT1 expression and/or the patient's pre-immune responses against WT1 may be assessed in a first step for example by companion diagnostics. Methods for assessing the expression of a target gene, such as WT1, either on mRNA or on protein level are well known to any one of ordinary skill in the art. For instance, immunohistochemistry staining, flow cytometry methods or RNA sequencing, or alternative methods using labelling can be used to identify the level of target expression in the tumor. Similarly, methods for assessing a patient's pre-immune response against a given protein, such as WT1, are well known to any one of ordinary skill in the art. A patient's pre-existing WT-1 specific T-cell pool can be detected by e.g. ELISpot or multimer FACS analysis. High WT1 expression and/or the occurrence of pre-immune responses against WT1 are prognostic indicators for the predisposition of a patient to respond favorably to the treatment with the attenuated strain of Salmonella encoding WT1, either alone or in combination with at least one checkpoint inhibitor.

It may be favorable dependent on the occurrence of possible side effects, to include treatment with antibiotics or anti-inflammatory agents.

Should adverse events occur that resemble hypersensitivity reactions mediated by histamine, leukotrienes, or cytokines, treatment options for fever, anaphylaxis, blood pressure instability, bronchospasm, and dyspnoea are available. Treatment options in case of unwanted T-cell derived auto-aggression are derived from standard treatment schemes in acute and chronic graft vs. host disease applied after stem cell transplantation. Cyclosporin and glucocorticoids are proposed as treatment options.

In the unlikely case of systemic Salmonella typhi Ty21a type infection, appropriate antibiotic therapy is recommended, for example with fluoroquinolones including ciprofloxacin or ofloxacin. Bacterial infections of the gastrointestinal tract are to be treated with respective agents, such as rifaximin.

In a further aspect, the present invention relates to a pharmaceutical composition comprising an attenuated strain of Salmonella comprising at least one copy of a DNA molecule comprising an expression cassette encoding WT1 for use in the treatment of cancer, wherein the treatment further comprises the administration of at least one checkpoint inhibitor. Particularly, the at least one checkpoint inhibitor is selected from at least one antibody against PD-1, PD-L1, CTLA-4, IDO, OX-40, GITR, TIM-3, and LAG-3. Preferably the at least one checkpoint inhibitor is at least one antibody against PD-1, PD-L1 or CTLA-4, or a combination thereof, more preferably the at least one checkpoint inhibitor is at least one antibody against PD-L1 or CTLA-4, or a combination thereof.

The pharmaceutical composition may be in the form of a solution, a suspension, an enteric coated capsule, a lyophilized powder or any other form suitable for the intended use.

The pharmaceutical composition may further comprises one or more pharmaceutically acceptable excipients.

In the context of the present invention, the term “excipient” refers to a natural or synthetic substance formulated alongside the active ingredient of a medication. Suitable excipients include antiadherents, binders, coatings, disintegrants, flavors, colors, lubricants, glidants, sorbents, preservatives and sweeteners.

In the context of the present invention, the term “pharmaceutically acceptable” refers to molecular entities and other ingredients of pharmaceutical compositions that are physiologically tolerable and do not typically produce untoward reactions when administered to a mammal (e.g., human). The term “pharmaceutically acceptable” may also mean approved by a regulatory agency of a Federal or a state government or listed in the U.S. Pharmacopeia or other generally recognized pharmacopeia for use in mammals, and, more particularly, in humans.

In particular embodiments, the pharmaceutical composition is provided as drinking solution. This embodiment offers the advantage of improved patient compliance and allows for rapid, feasible and affordable mass vaccination programs.

In particular, suitable drinking solutions comprise means to neutralize gastric acids to at least to a certain degree, i.e. to bring the pH of the gastric juice closer to a pH of 7. In a particular embodiment, the drinking solution is a buffered suspension obtained by suspending the attenuated strain of Salmonella according to the present invention in a suitable buffer, preferably in a buffer that neutralizes gastric acids to at least a certain degree, preferably in a buffer containing 2.6 g sodium hydrogen carbonate, 1.7 g L-ascorbic acid, 0.2 g lactose monohydrate and 100 ml of drinking water.

In particular embodiments, the attenuated strain of Salmonella is Salmonella typhi Ty21 a.

In particular embodiments, the expression cassette is a eukaryotic expression cassette.

In particular embodiments, WT1 is selected from the group consisting of WT1 having the amino acid sequence as found in SEQ ID NO 1 and a protein that shares at least about 80% sequence identity therewith.

Particularly, the eukaryotic expression cassette comprises a CMV promoter.

Particularly, WT1 has the amino acid sequence as found in SEQ ID NO 1.

In particular embodiments, the treatment comprises a single or multiple administrations of the attenuated strain of Salmonella encoding WT1 or a pharmaceutical composition comprising the same and/or the at least one checkpoint inhibitor. The single dose of the administrations may be the same or different. In particular, the treatment comprises 1, 2, 3, 4, 5 or 6 administrations of the attenuated strain of Salmonella encoding WT1 and/or the at least one checkpoint inhibitor, preferably wherein the multiple administrations occur within three to six consecutive months.

### SHORT DESCRIPTION OF FIGURES AND TABLES

FIG. 1: Amino acid sequence of human WT1, wherein the zinc finger domain is deleted (SEQ ID NO 1), which is encoded by WT1 cDNA contained in plasmid pVAX10.hWT1

FIG. 2: Nucleic acid sequence comprised in empty expression vector pVAX10 (sequence of expression vector pVAX10 without the portion of the multiple cloning site which is located between the restriction sites NheI and XhoI (SEQ ID NO 2).

FIG. 3: Nucleic acid sequence contained in plasmid pVAX10.hWT1 and encoding human WT1 of SEQ ID NO 1

FIG. 4: Amino acid sequence of human full length WT1, UniProt ref P19544-7 (SEQ ID NO 4)

FIG. 5: Amino acid sequence of human MSLN encoded by MSLN cDNA contained in plasmid pVAX10.hMSLN (SEQ ID NO 5)

FIG. 6: Amino acid sequence of human CEA encoded by CEA cDNA contained in plasmid pVAX10.hCEA (SEQ ID NO 6)

FIG. 7: Amino acid sequence of CMV pp65 encoded by CMV pp65 cDNA contained in plasmid pVAX10.CMVpp65_1 (SEQ ID NO 7)

FIG. 8: Amino acid sequence of CMV pp65 encoded by CMV pp65 cDNA contained in plasmid pVAX10.CMVpp65_2 (SEQ ID NO 8)

FIG. 9: Amino acid sequence of CMV pp65 encoded by CMV pp65 cDNA contained in plasmid pVAX10.CMVpp65_3 (SEQ ID NO 9)

FIG. 10: Amino acid sequence of VEGFR-2 encoded by VEGFR-2 cDNA contained in plasmid pVAX10.VR2-1 (SEQ ID NO 10)

FIG. 11: Plasmid map of pVAX10.hWT1

FIG. 12: Effects of the combined administration of VXM06m with anti-CTLA-4 on the survival of C57BL/6 mice bearing disseminated FBL-3

FIG. 13: Effects of the combined administration of VXM06m with anti-PD-L1 on the survival of C57BL/6 mice bearing disseminated FBL-3

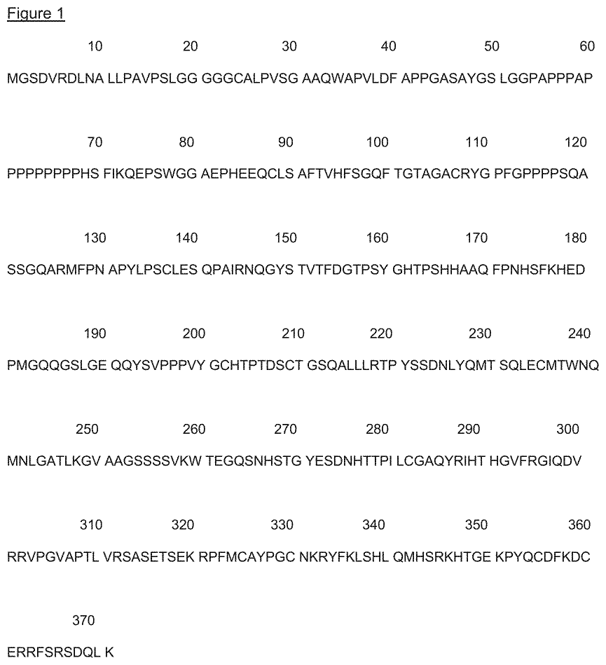

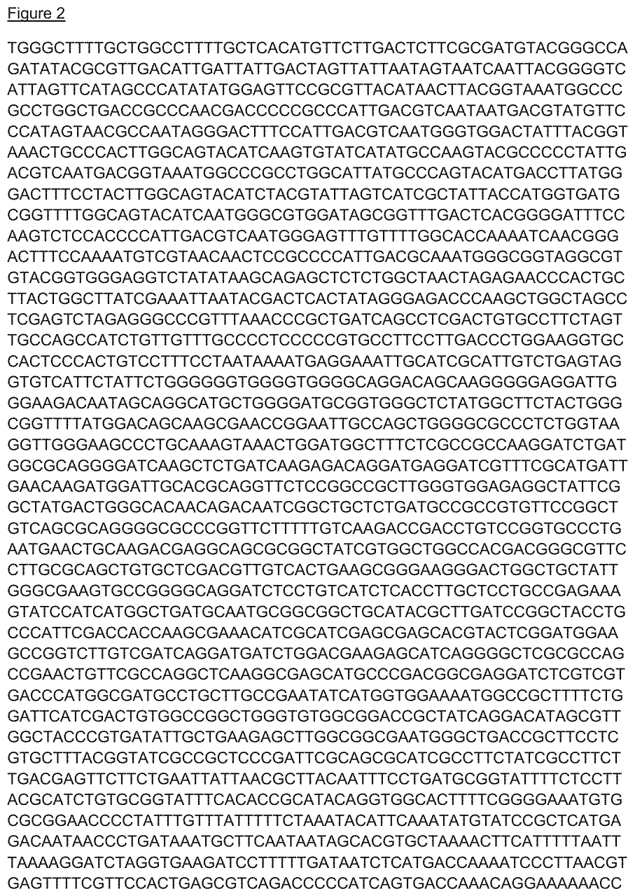

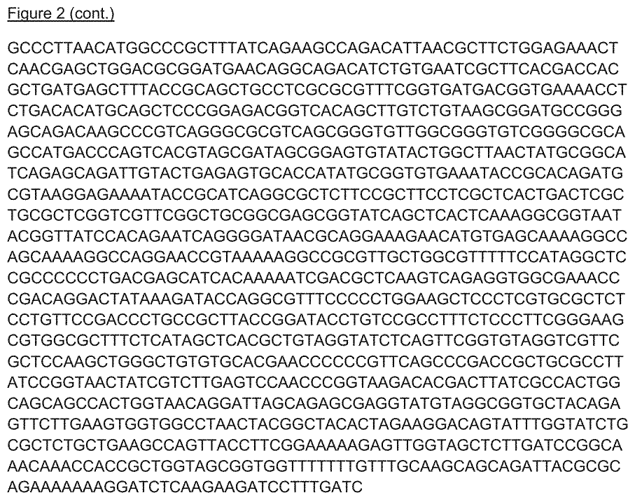

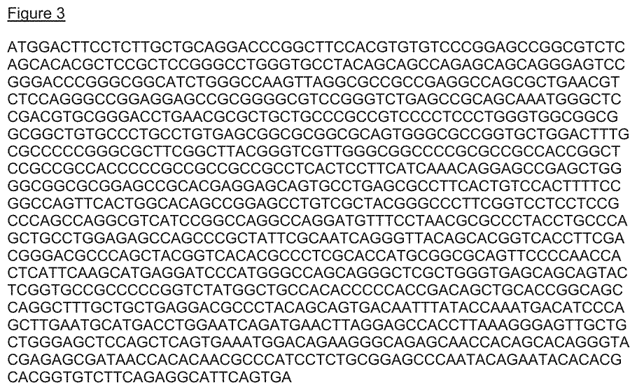

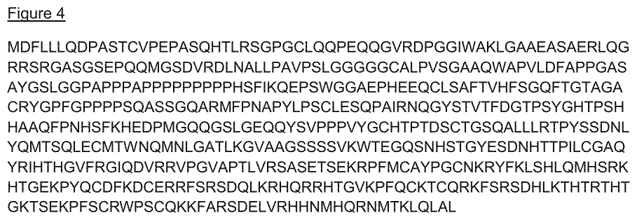

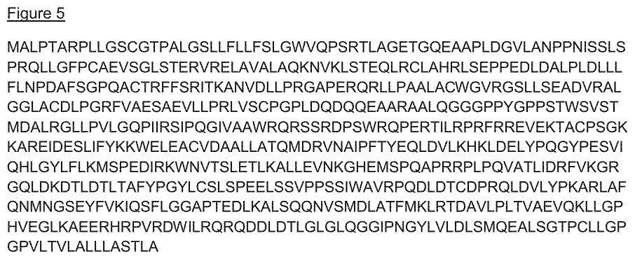

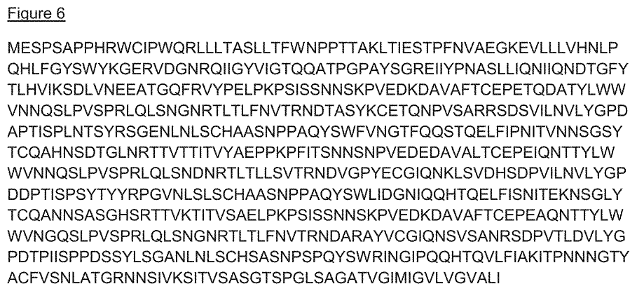

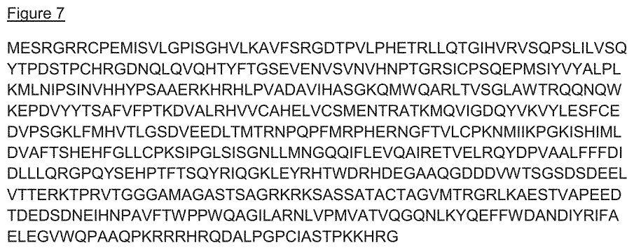

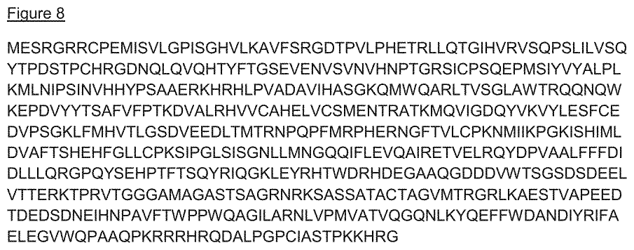

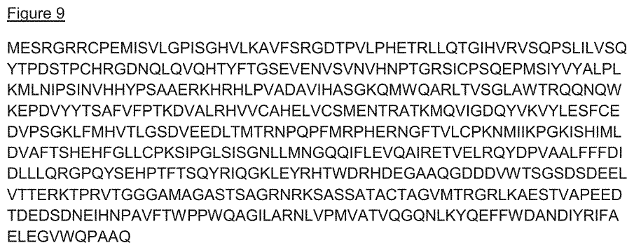

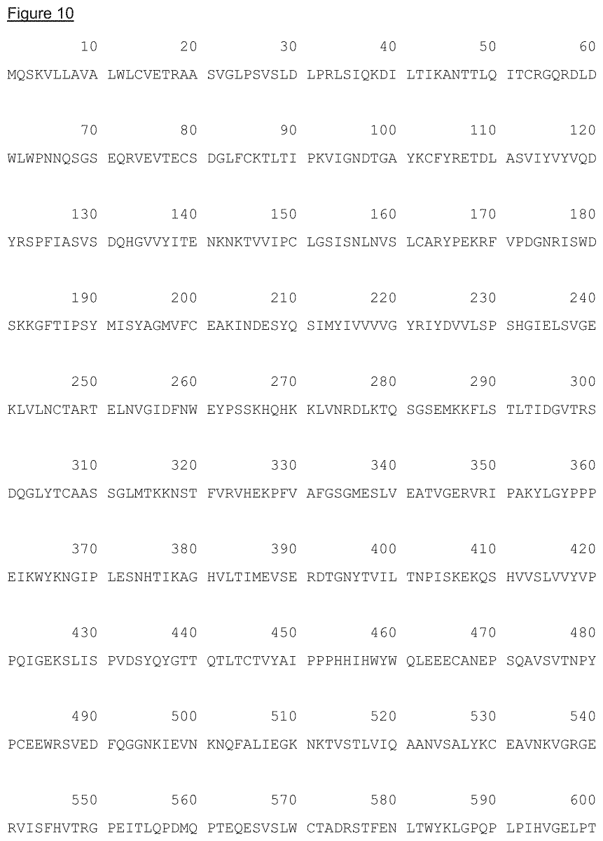

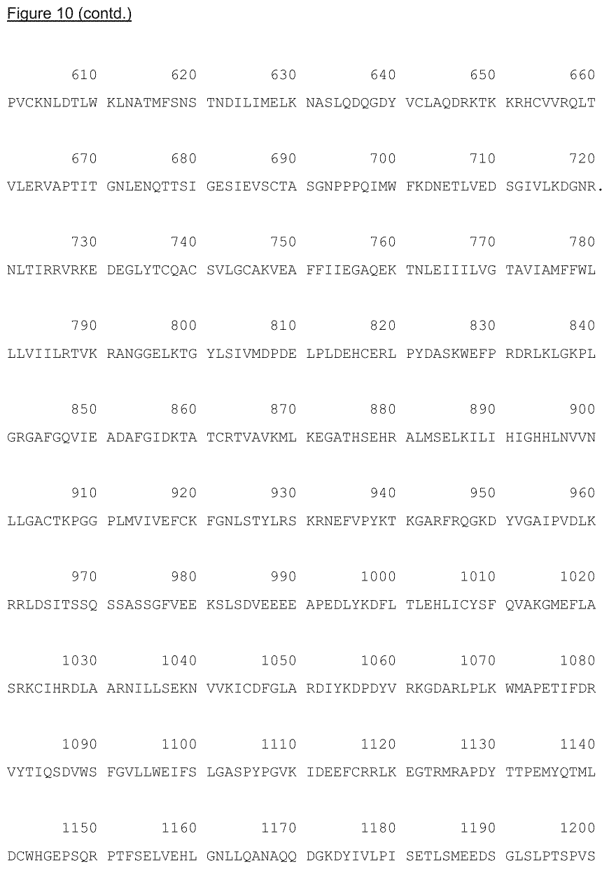

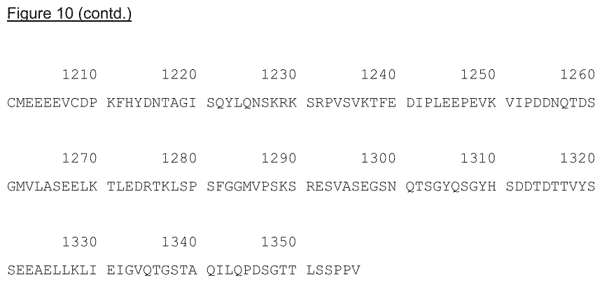

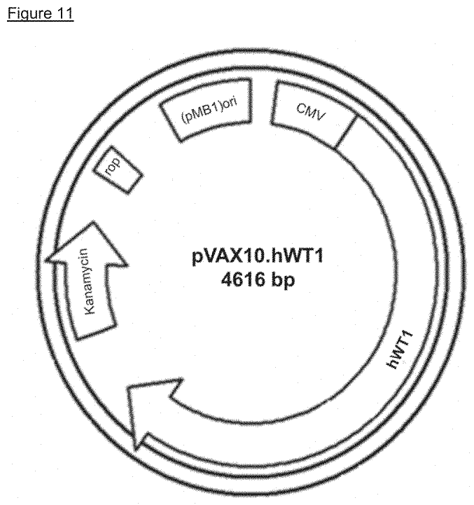

### EXAMPLES

Assessment of the Antitumor Activity of VXM06m in Combination with PD-L1 and CTLA-4 Checkpoint Inhibitor Blockade:

The aim of this study was to investigate the antitumor activity of VXM06m in combination with PD-L1 and CTLA-4 checkpoint inhibitor blockade in a syngeneic tumor model of leukemia induced by the mouse cell line FBL-3 intraperitoneally implanted in C57BL/6 mice.

60 male C57/BL6 mice, 4-6 weeks old, with an average weight of about 20 g/mouse, were randomized into 4 groups of 15 animals each.

Group 1 (control group): The mice (n=15) were treated with the empty vector VXM0m_empty (S. typhimurium bacterial vector control harboring no exogenous expression plasmid) at days, 1, 3, 5, 7, 14 and 22. At day 20, FBL-3 tumor cells were implanted in mice by I.P. route.

Group 2: The mice (n=15) were treated with VXM06m (S. typhimurium containing pVAX10.mWT1 coding for truncated murine WT1) at a dose of 10⁸ CFU/application at days, 1, 3, 5, 7, 14 and 22. Meanwhile, the mice were treated with 100 μg/application of anti-PD-L1 monoclonal antibody (BP0101, In Vivo Plus anti mouse PD-L1, Clone 10F.9G2, BioXCell) at days 24 and 29 by I.P. route. At day 20, FBL-3 tumor cells were implanted in mice by I.P. route.

Group 3: The mice (n=15) were treated with VXM06m at a dose of 10⁸ CFU/application at days, 1, 3, 5, 7, 14 and 22. Meanwhile, the mice were treated with 100 μg/application of anti-CTLA-4 monoclonal antibody (Anti CTLA-4 antibody, clone UC10-4F10, BioXCell) at days 11 and 18, by I.P. route. At day 20, FBL-3 tumor cells were implanted in mice by I.P. route.

Group 4 (control group): The mice (n=15) were treated with the empty vector VXM0m_empty (10⁸ CFU/application) at days, 1, 3, 5, 7, 14 and 22 and with anti-CTLA-4 (100 μg/dose) at days 11 and 18. At day 20, FBL-3 tumor cells were implanted in mice by I.P. route.

FBL-3 is a Friend leukemia virus-induced erythroleukemia cell line originated from C57BL/6 mice. This cell line expresses unique TSTA (Tumor Specific Transplantation Antigens) that can be recognized by the immune system. Priming syngeneic mice with FBL-3 tumor cells leads to the subsequent rejection of future live tumor challenges. Although FBL-3 is immunogenic, injection of live FBL-3 tumor cells into naive syngeneic mice results in tumor growth, suggesting that the FBL-3 tumor cells possess mechanisms to escaping immune recognition and destruction. Importantly, FBL-3 has been shown to overexpress Wilms tumor 1 (WT1).

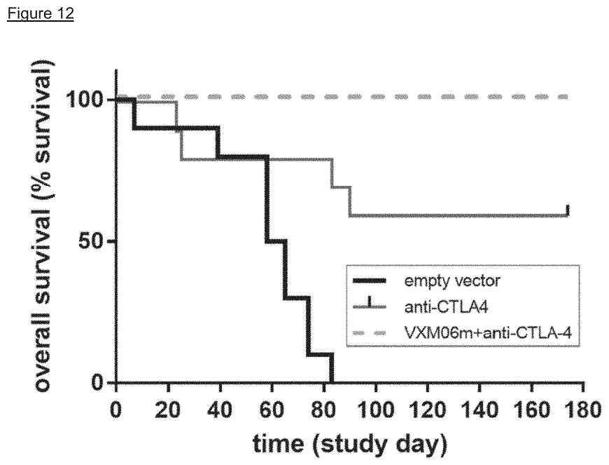

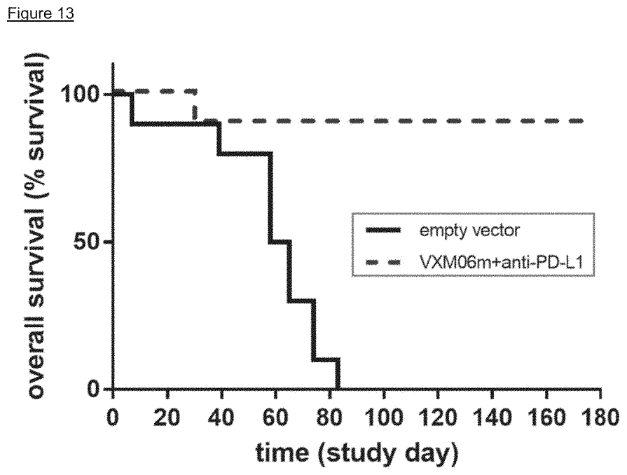

Mice survival time was carefully monitored throughout the whole study and is illustrated in FIGS. 12 and 13.

One mouse death was observed in group 1 treated with empty vector on day 7 probably due to the accidental perforation of the esophagus the day of vaccine administration.
On Day 23 one mouse was dead in group 4.
On Day 25 one mouse was dead in group 4.
On day 29; 5 mice of each group were sacrificed and spleens were collected.
On day 30, one mouse was dead in group 2.
On day 39, one mouse was dead in group 1.
On day 58, 3 mice were dead in group 1.
On day 65, 2 mice were dead in group 1.
On day 74, 2 mice were dead in group 1.
On day 83, 2 mice were dead in group 1 & 4.
On day 90, one mouse was dead in group 4.

Thus, after the first tumor cell challenge at day 20, animal mortality was observed only in groups 1, 2 and 4, and for the other groups, all animals were healthy and no tumor development was observed.

These results clearly indicate that the combination of VXMO6m with either of the checkpoint inhibitors anti-CTLA-4 and anti-PD-L1 is highly effective in the FBL-3 model, generating a rapid and sustained anti-tumor effect. The VXMO6m plus anti-CTLA-4 combination led to 100% survival at the end of study (day 196).

In contrast, treatment with empty vector did not show anti-tumor effect, with a mean survival of 45 days after tumor challenge and 0% recovery. Treatment with anti-CTLA-4 led to 40% death at the end of study.

1. A method for treating cancer in a subject, comprising administering an attenuated strain of Salmonella comprising at least one copy of a DNA molecule comprising an expression cassette encoding Wilms' Tumor Protein (WT1), wherein the method further comprises the administration of at least one checkpoint inhibitor.

2. The method according to claim 1, wherein the checkpoint inhibitor is selected from at least one antibody against PD-1, PD-L1, CTLA-4, IDO, OX-40, GITR, TIM-3, and LAG-3.

3. The method according to claim 1, wherein the attenuated strain of Salmonella is of the species Salmonella enterica, particularly wherein the attenuated strain of Salmonella is Salmonella typhi Ty21a.

4. The method according to claim 1, wherein the expression cassette is a eukaryotic expression cassette, particularly wherein the expression cassette comprises a CMV promoter.

5. The method according to claim 1, wherein
(a) WT1 is selected from the group consisting of human WTI having the amino acid sequence as found in SEQ ID NO 4 and a protein that shares at least about 80% sequence identity therewith,
(b) wherein WT1 is truncated, more particularly wherein the zinc finger domain of WT1 is deleted, or
(c) wherein WT1 is selected from the group consisting of WT1 having the amino acid sequence as found in SEQ ID NO 1 and a protein that shares at least about 80% sequence identity therewith.

6. The method according to claim 1, wherein the DNA molecule comprises the kanamycin antibiotic resistance gene, the pMB1 ori, and a CMV promoter, particularly wherein the DNA molecule comprises the DNA sequence as found in SEQ ID NO 2.

7. The method according to claim 1, wherein the attenuated strain of Salmonella is administered simultaneously with, prior to or after said at least one checkpoint inhibitor.

8. The method according to claim 1, wherein the treatment is accompanied by chemotherapy, radiotherapy or biological cancer therapy, particularly wherein the attenuated strain of Salmonella is administered before, during or after the chemotherapy or the radiotherapy treatment cycle or the biological cancer therapy, or before and during the chemotherapy or the radiotherapy treatment cycle or the biological cancer therapy.

9. The method according to claim 8, wherein the biological cancer therapy comprises administration of one or more further attenuated strain(s) of Salmonella comprising at least one copy of a DNA molecule comprising an expression cassette encoding a tumor antigen and/or a tumor stroma antigen, particularly wherein said one or more further attenuated strain(s) of Salmonella is/are Salmonella typhi Ty21a comprising a eukaryotic expression cassette

10. The method according to claim 9, wherein said tumor antigen is selected from the group consisting of Mesothelin (MSLN), CEA, and CMV pp65 and/or said tumor stroma antigen is selected from the group consisting of VEGF receptor protein and human fibroblast activation protein (FAP).

11. The method according to claim 10, wherein said tumor antigen is selected from the group consisting of MSLN having the amino acid sequence as found in SEQ ID NO 5 and a protein that shares at least about 80% sequence identity therewith, CEA having the amino acid sequence as found in SEQ ID NO 6 and a protein that shares at least about 80% sequence identity therewith, CMV pp65 having the amino acid sequence as found in SEQ ID NO 7 and a protein that shares at least about 80% sequence identity therewith, CMV pp65 having the amino acid sequence as found in SEQ ID NO 8 and a protein that shares at least about 80% sequence identity therewith, and CMV pp65 having the amino acid sequence as found in SEQ ID NO 9 and a protein that shares at least about 80% sequence identity therewith, and/or
wherein said tumor stroma antigen is selected from the group consisting of a VEGFR-2 having the amino acid sequence as found in SEQ ID NO 10 and a protein that shares at least about 80% sequence identity therewith, and human fibroblast activation protein (FAP).

12. The method according to claim 1, wherein the attenuated strain of Salmonella is administered orally.

13. The method according to claim 1, wherein the cancer is selected from leukemia, particularly from acute myeloid leukemia (AML) and acute lymphoid leukemia (ALL), from multiple myeloma, and from solid tumors, particularly from lung cancer, breast cancer, esophageal, colon, colorectal, gastric, cholangioductal, pancreatic cancer, glioblastoma, head and neck cancer, synovial sarcoma, angiosarcoma, osteosarcoma, thyroid cancer, cervical, endometrial, ovarian cancer, neuroblastoma, rhabdomyosarcoma, and prostate cancer.

14. The method according to claim 1, wherein the single dose of the attenuated strain of Salmonella comprises from about 10⁵ to about 10¹¹, particularly from about 10⁶ to about 10¹⁰, more particularly from about 10⁶ to about 10⁹, more particularly from about 10⁶ to about 10⁸, most particularly from about 10⁶ to about 10⁷ colony forming units (CFU).

15. The method according to claim 1, wherein the treatment is individualized cancer immunotherapy comprising the step of assessing the WT1 expression and/or the pre-immune response against WT1 in a patient.

16. A method for treating cancer in a subject, comprising administering a pharmaceutical composition comprising an attenuated strain of Salmonella comprising at least one copy of a DNA molecule comprising an expression cassette encoding WT1, wherein the method further comprises the administration of at least one checkpoint inhibitor, particularly selected from an antibody against PD-1, PD-L1, CTLA-4, IDO, OX-40, GITR, TIM-3, and LAG-3.

17. The method according to claim 16, wherein the attenuated strain of Salmonella is Salmonella typhi Ty21a, wherein the expression cassette is a eukaryotic expression cassette, particularly comprising a CMV promoter, and wherein WT1 is selected from the group consisting of human WT1 having the amino acid sequence as found in SEQ ID NO 1 and a protein that shares at least about 80% sequence identity therewith, particularly wherein human WT1 has the amino acid sequence as found in SEQ ID NO 1.
